(19) United States
(12) Patent Application Publication
(10) Pub. No.: US 2011/0236445 A1
(43) Pub. Date: Sep. 29, 2011

(54) MOUTHWASH IN THE FORM OF A STABLE SUSPENSION COMPRISING MICROSPHERES THAT INCORPORATE AN ACTIVE INGREDIENT

(75) Inventors: Francesco Cilurzo, Gaggiano MI (IT); Paola Minghetti, Gaggiano MI (IT)

(73) Assignee: PHARMAFILM S.R.L., Gaggiano MI (IT)

(21) Appl. No.: 13/129,624

(22) PCT Filed: Nov. 23, 2009
(86) PCT No.: PCT/EP09/65669

§ 371 Date: May 17, 2011

(30) Foreign Application Priority Data
Nov. 25, 2008 (IT) MI2008A002104

(51) Int. Cl. A61K 8/02; A61K 8/81; A61Q 11/00

(52) U.S. Cl. 424/401; 424/49

(57) ABSTRACT

The present invention relates to a mouthwash including microspheres that incorporate an active ingredient, wherein the microspheres are obtained directly in situ in the aqueous phase of the mouthwash by cross-linking. These microspheres do not tend to aggregate and the mouthwash appears in the form of a homogenous and stable suspension even after prolonged storage.

### FIELD OF THE INVENTION

The present invention relates to a mouthwash in the form of a stable suspension comprising microspheres that incorporate an active ingredient.

### STATE OF THE ART

The microencapsulation of active ingredients is a very useful method to prolong drug delivery and reduce side effects. The generic definition of microparticles includes microspheres, which represent a very important delivery form because of their small size and very efficient carrying properties. However, the success of microspheres is limited by the fact that they have a very low residence time at the absorption site. To obviate this problem “Design and Development of Gliclazide Mucoadhesive Microcapsules: In Vitro and In Vivo Evaluation” S. K. Prajapati, Purnima Tripathy, Udhumansha Ubaidulla and Vikas Anand, AAPS Pharm. Sc. Tech, Vol. 9 No. 1 Mar. 2008 224-230 disclose the preparation of microspheres, ranging in size between 1 μm and 1000 μm, that incorporate gliclazide (an active ingredient used in the treatment of type II diabetes) and are prepared using sodium alginate in combination with one of the following polymers: sodium carboxymethylcellulose, hydroxypropylmethylcellulose, and a cross-linked polyacrylate, like Carbopol 934. In a first step, an aqueous polymer dispersion of sodium alginate and one of the three above-mentioned polymers is prepared and the active ingredient is added thereto. In a second step, the aqueous dispersion of polymers and active ingredient is added to an aqueous solution comprising a salt of an alkali metal, like calcium chloride, able to cross-link the alginate thereby forming rigid microspheres. The microspheres are removed from the mother liquors by decantation, washed with water, and then desiccated, and are generally used for the preparation of capsules and tablets; however, their use in the preparation of mouthwashes is not known.

Particles with a size of about 1 mm able to incorporate toothpaste aromatizers are disclosed in WO 99/24159. This prior art discloses also the preparation of mouthwashes containing said particles, in which the particles are formed in situ in the mouthwash liquid phase, but in the majority of cases the particles are separated from the formation bath, washed, and then dispersed again in fresh mouthwash liquid phase for storage. The Applicant noted mouthwashes prepared according to this prior art are not stable, because the particles, especially micron-size particles not filtered off the mouthwash liquid phase, tend to form aggregates under storage conditions.

There is therefore a demand for mouthwashes comprising microparticles having low tendency to form aggregates and at the same time high surface area and high mucoadhesivity.

### SUMMARY OF THE INVENTION

The Applicant has now found out that it is possible to obviate the above-mentioned inconveniences with the mouthwash according to the present invention.

In particular, the mouthwash according to the present invention contains microspheres incorporating an active ingredient (c) and is characterized in that the microspheres are obtained in situ by adding drops of size smaller than 700 μm of an aqueous polymer dispersion (A), containing:
(a) polyacrylic acid, optionally cross-linked and optionally in the form of a salt with an alkali metal and
(b) an alkali metal alginate
   to an aqueous solution (B) (herein after also referred to as “cross-linking bath”) comprising a pharmaceutically acceptable organic or inorganic salt of an alkaline earth metal, able to cross-link the alginate,
   wherein either dispersion (A) or aqueous solution (B) or both may contain active ingredient (c). The choice of adding the active ingredient to dispersion (A), aqueous solution (B) or both depends on the active ingredient and on its chemico-physical properties. For example, active ingredients like delmopinol hydrochloride and chlorexidine gluconate are preferably added to the cross linking bath.

The Applicant has indeed found out that the microparticles comprised in the mouthwash of the present invention have very low tendency to aggregate and also optimal mucoadhesivity properties, as demonstrated in the examples reported below.

### DISCLOSURE OF THE INVENTION

Preferably, the aqueous polymer dispersion (A) in the mouthwash according to the present invention has a polymer total content in polymers (a) and (b) ranging from 0.5 to 5% by weight with respect to the total weight of the aqueous dispersion. Preferably, the alkali metal alginate (b)/optionally cross-linked polyacrilate (a) weight ratio in the aqueous dispersion (A) ranges from 1:1 to 14:1, more preferably from 1:1 to 9:1.

The alkaline alginate (b) is preferably sodium alginate; for the purposes of the present invention it is preferable to use an alginate with viscosity ranging from 10 to 400 mPA·s as measured in a solution containing said alginate in 1% concentration at 20° C. According to a particularly preferred embodiment, commercial products selected among Protanal LF120M, Protanal LF200M, and Protanal LF10/60 are used.

The polyacrylic acid (a) can be either cross-linked or non cross-linked and is preferably used in the form of a salt with sodium salt. According to a particularly preferred embodiment, the products commercially available under the names Carbopol 940 and Carbopol 971 are used as non cross-linked sodium polyacrylates, whereas the most preferred cross-linked products are commercially available under the commercial names Carbopol 934 P and Carbopol 974. Of particular interest is also polycarbophil, i.e. polyacrylate cross-linked with divinyl glycol, commercially available under the trade name of Noveon AA-1. The presence of cross-linked or non cross-linked polyacrylic acid is critical in order to obtain a low tendency to aggregate; indeed, microspheres containing other polymers like sodium carboxymethylcellulose, hyaluronic acid, or hydroxypropylmethylcellulose instead of polyacrylates have a very high tendency to aggregate, as demonstrated by the Applicant in the examples reported below.

The salt of the alkaline earth metal usable for preparing the cross-linking bath is an organic or inorganic calcium or barium salt. Preferably, it is a calcium salt selected between chloride and lactate. The latter is particularly preferred in that it leaves a more pleasant taste in the mouth.

Preferably, in the mouthwash according to the present invention, polymer dispersion (A) is added in the form of microdrops to the cross-linking bath comprising the salt of the alkaline earth metal, until concentrations ranging from 5% to 25% by weight with respect to the total mouthwash weight, more preferably from 10% to 15%, are reached.

The size of the microspheres is preferably smaller than 700 μm. According to a particularly preferred embodiment, the particle size distribution of the microspheres present in the mouthwash, measured as volume d₉₀, ranges from 50 to 500 μm, more preferably from 100 to 300 μm.

The active ingredient is preferably selected from: local anaesthetics, nonsteroidal antiinflammatory drugs, corticosteroids, antiseptics, anticaries agents, local anaesthetics, cicatrizants.

More preferably, the active principle is selected from the group consisting of: chlorhexidine, alexidine, octenidine, hexetidine fluoride, thymol, hexylresorcinol, cetylpyridinium chloride, benzethonium chloride, delmopinol hydrochloride, dequalinium, bloodroot, rhubarb, benzydamine, ketoprofen, flurbiprofen, ibuprofen, methylprednisolone, clobetasol propionate, triamcinolone acetonide, salicylic acid, lidocaine, procaine, and pharmaceutically acceptable salts thereof.

The microspheres in the mouthwash of the present invention preferably comprise the active ingredient in amounts ranging from 0.01 to 5%, preferably from 0.1 to 1% by weight with respect to the total weight of the microspheres. However, in the mouthwash of the invention, part of the active ingredient is also present in the liquid phase; this ensures prolonged release of the active ingredient, because the active ingredient contained in the liquid phase is immediately delivered to the oral mucosa, while the active ingredient contained in the microspheres is released slowly.

The mouthwash of the present invention is preferably prepared with a process comprising the following steps:

i) dispersing in water the optionally cross-linked polyacrylic acid or an alkaline salt thereof (a) and the alkali metal alginate (b) in order to obtain the aqueous polymer dispersion (A);

ii) spraying drops of size smaller than 700 μm of the aqueous polymer dispersion (A), obtained in the previous step, in the aqueous solution (B) of an organic or inorganic salt of an alkali metal able to cross-link the alginate

wherein the active ingredient (c) is added either to the polymer dispersion (A) or to the aqueous solution (B) or to both dispersion (A) and solution (B).

The mouthwash of the present invention can optionally comprise other active ingredients and excipients commonly used in mouthwashes, for example preservatives, sweeteners, aromatizers, cosolvents (ethanol, glycerin, polyethyleneglycoles, etc).

The following examples illustrate the preparation of mouthwashes according to the invention and show their improved features, with special regard to their low tendency to form aggregates.

### EXAMPLE 1

### Placebo Mouthwashes

Placebo mouthwashes were prepared in one-step by spraying polymer dispersions (A) into calcium chloride cross-linking baths, as described below in detail.

### Preparation of Polymer Dispersions

Polymers (an alginate and a polyacrylate) were dispersed in water by mixing until clear and homogeneous dispersions 1-16 having the compositions reported in table 1 were obtained.

TABLE 1
Polymer dispersions compositions (% w/w)

| Disper | Protanal | Protanal | Protanal | | | | | | HPMC | | | |
|---|---|---|---|---|---|---|---|---|---|---|---|---|
| n. | LF120 | LF200 | LF10/60 | C934P* | C940** | C971*** | C974**** | Polycarbophyl | K4M | CMC | HA | Water |
| 1 | 3 | | | | | | | | | | | 97 |
| 2 | 1.5 | | | 0.5 | | | | | | | | 98 |
| 3 | 1.5 | | | 1.5 | | | | | | | | 97 |
| 4 | 3 | | | 0.5 | | | | | | | | 96.5 |
| 5 | | 1 | | 0.5 | | | | | | | | 98.5 |
| 6 | | | 2 | 0.5 | | | | | | | | 97.5 |
| 7 | 1.5 | | | | 0.5 | | | | | | | 98 |
| 8 | 1.5 | | | | | | | | 0.5 | | | 98 |
| 9 | 1.5 | | | | | | | | | 0.5 | | 98 |
| 10 | 1.5 | | | | | | | | | | 0.4 | 98.1 |
| 11 | 2.7 | | | 0.3 | | | | | | | | 97 |
| 12 | 2.8 | | | 0.2 | | | | | | | | 97 |
| 13 | 1.5 | | | 1 | | | | | | | | 97.5 |
| 14 | 1.5 | | | | | 1.5 | | | | | | 97 |
| 15 | 1.5 | | | | | | 1.5 | | | | | 97 |
| 16 | 1.5 | | | | | | | 1.5 | | | | 97 |

Notes:
Protanal: sodium alginate;
*Carbopol 934P,
**Carbopol 940,
***Carbopol 971,
****Carbopol 974 and polycarbophyl: carboxypolymethylene; HPMC K4M: hydroxypropylmethylcellulose; CMC: sodium carboxymethylcellulose; HA hyaluronic acid

The dispersions were let to rest for 12 hours in order to remove any entrapped air.

### Mouthwashes Preparation

80 mL of the dispersions 1-16 were sprayed through a standard two-way nozzle (0.8 mm inner diameter) into a calcium chloride cross-linking bath under magnetic stirring. The resulting mouthwashes, whose compositions are reported in table 2 below, were kept under stirring for 1 hour in order to allow complete cross-linking of alginate with calcium; formation of microspheres precipitates that could be easily dispersed by simple agitation was observed. The mouthwashes (1-21) were then filled in 200 ml bottles and stored at 25° C. The compositions of the placebo mouthwashes is reported in table 2 below.

TABLE 2
Compositions of the placebo mouthwashes

| Mouthwash | Dispersion* | | CaCl₂ | Water |
|---|---|---|---|---|
| n. | n. (g) | | (g) | (g) |
| 1 | 1 | 80 | 16 | 704 |
| 2 | 2 | 80 | 16 | 704 |
| 3 | 2 | 80 | 4 | 716 |
| 4 | 2 | 240 | 4 | 756 |
| 5 | 2 | 80 | 4 | 716 |
| 6 | 3 | 80 | 16 | 704 |
| 7 | 3 | 80 | 4 | 716 |
| 8 | 4 | 80 | 4 | 716 |
| 9 | 4 | 160 | 4 | 612 |
| 10 | 5 | 80 | 16 | 704 |
| 11 | 6 | 80 | 16 | 704 |
| 12 | 7 | 80 | 16 | 704 |
| 13 | 8 | 80 | 16 | 704 |
| 14 | 9 | 80 | 16 | 704 |
| 15 | 10 | 80 | 16 | 704 |
| 16 | 11 | 160 | 4 | 704 |
| 17 | 12 | 80 | 4 | 716 |
| 18 | 13 | 80 | 16 | 704 |
| 19 | 14 | 80 | 16 | 704 |
| 20 | 15 | 80 | 16 | 704 |
| 21 | 16 | 80 | 16 | 704 |

### 1.2 Determination of Aggregates Formation

After one-week storage, the mouthwashes were stirred in order to re-suspend the microspheres precipitates, then 50 mL aliquots were taken and filtered through a sieve (sieve opening: 710 μm). The sieve was dried and the presence of microspheres aggregates was evaluated by measuring the difference between the sieve weight before and after filtration. The assay requirements were complied with (i.e. no aggregates were deemed to be present) when sieve weight variation was lower than 8 mg.

Results:

### Mouthwash 1: 0.445 g

### Mouthwash 13: 0.319 g

### Mouthwash 14: 0.303 g

### Mouthwash 15: 0.205 g

Weight variations lower than 8 mg were observed also for all other mouthwashes.

The assay was repeated with mouthwashes n. 2 and 3 after three-month storage at 25° C. and 40° C.; the assay requirements were met for both samples.

### 1.3 Determination of Microsphere Particle Size

Mouthwashes 1, 2 and 6 were filtered and suitably diluted with water.

The mean microsphere diameter was determined by using a particle size analyzer (Accusizer TM 780A). The results, expressed in micrometers, are reported in table 3 below:

TABLE 3
Microsphere particle size

| | Area | Volume | | |
|---|---|---|---|---|
| | weighted | weighted | | |
| | mean | mean | d₉₀ | d₉₀ |
| Mouthwash | diameter | diameter | (area) | (volume) |
| 1 | 51 | 101 | 128 | 148 |
| 2 | 54 | 81 | 102 | 125 |
| 6 | 71 | 96 | 125 | 133 |

### 1.4 Mucoadhesion Properties

Mouthwashes 1, 6, 18-20 were filtered through a sieve (sieve opening: 90 μm). 100 mg aliquots of microspheres were taken, placed in 15 mL falcon tubes and suspended in 2.5 mL water. A 2.5 ml mucin solution (0.05% w/v) was added to each sample. The samples were incubated at 37° C. for 5 minutes at 100 rpm and then centrifuged at 1000×g for 30 seconds. After discharging the supernatants, fresh water (5 mL) was added to each tube to remove mucin adsorbed on the microparticle surface. This water was discharged and the microspheres were re-suspended in 2.5 mL fresh water. 200-μl aliquots of each suspension were taken and assayed for the bound mucin with a micro BCA reagent kit; the samples were read with a Beckman DU-65 spectrophotometer at 562 nm after 30 minutes incubation at 37° C., following a method adapted from Lei Shil and Karin D. Caldwell, Mucin Adsorption to Hydrophobic Surfaces. Journal of Colloid and Interface Science, 224, 372-381 (2000).

### Results

The results, expressed as milligrams of bound mucin per gram of microspheres, are reported in table 4 below:

TABLE 4
Bound mucin per
gram of microspheres

| Mouthwash | Bound |
|---|---|
| n. | mucin |
| 1 | 1.334 ± |
| | 0.118 |
| 6 | 2.764 ± |
| | 0.153 |
| 18 | 2.012 ± |
| | 0.177 |
| 19 | 3.043 ± |
| | 0.192 |
| 20 | 2.223 ± |
| | 0.161 |

Mouthwashes 6, 18, 19, 20 are statistically different from mouthwash 1, indicating that the presence of carboxypolymethylene improves the mucoadhesive properties of the microparticles.

### EXAMPLE 2

### Mouthwash Containing Delmopinol Hydrochloride

### 2.1 Mouthwash Preparation

The compositions of the polymer dispersions and of the cross-linking baths, prepared according to example 1, are reported in Table 5 below.

TABLE 5
Compositions (grams) of the polymer dispersions
and of the cross-linking baths

| | Composition of the polymer | | | Composition of the | | |
|---|---|---|---|---|---|---|
| | dispersion | | | cross-linking bath | | |
| Mouthwash | Protanal | | | Dp | | |
| n. | LF120 | C934P* | Water | Cl | CaCl₂ | Water |
| 22 | 1.5 | 0.5 | 98.0 | 2.2 | 18.0 | 879.8 |
| 23 | 1.5 | 0.5 | 98.0 | 1.1 | 18.0 | 880.9 |

Notes:
Protanal: sodium alginate;
*Carbopol 934P; Dp Cl: delmopinol hydrochloride

### 2.2 Determination of Aggregates Formation

After one-week storage, the mouthwashes were stirred in order to re-suspend the microparticles, and then 50 mL mouthwashes were taken, filtered through a sieve (sieve opening: 710 μm) and tested for the presence of aggregates as described in example 1.2 above. The assay requirements were complied with (i.e. no aggregates were deemed to be present) when sieve weight variation was lower than 8 mg.

### Results

Both mouthwashes (22-23) complied with the assay requirements.

### 2.3 Evaluation of Delmopinol Content in the Mouthwashes Liquid Phase and in the Microspheres

a) Delmopinol encapsulated in the microspheres 20 ml mouthwash were filtered through a HVLP 0.45 μm membrane filter, and the microspheres on the filter were washed with 2 ml MeOH. The microspheres (about 50 mg) were dispersed in 10 ml sodium chloride (0.9% w/v) and digested by sonication for 1 hour. The resulting dispersions were filtered through a polypropylene 0.45 μm filter and analyzed by high-performance liquid chromatography (HPLC), according to the method described below.
b) Delmopinol in the mouthwashes liquid phase 2 mL aliquots of each mouthwash were filtered through 0.45 μm polypropylene filters and diluted 1:10 with HPLC water; these samples were assayed by HPLC according to the method described below.

### HPLC Method

HP1100 series, Chemstations Hewlett Packard, USA; Column: SymmetryShield™ RP18, 5 μm, 4.6 mm×150 mm (Waters, Ireland); flux: 1 ml/min; mobile phase: 800 mL MeOH+200 mL buffer pH 7.0; injection volume: 10 μl; wavelength: 210 nm.

### Results

In this and in the following examples, the results of the tests for the evaluation of the amount of active principle in the microspheres and in the liquid phase are expressed as weight percentage on the total weight of the microspheres and of the liquid phase respectively.

### Mouthwash n. 23

a) Delmopinol encapsulated in the microspheres: 0.2%
b) Delmopinol in the mouthwash liquid phase: 0.1%

### Mouthwash n. 22

a) Delmopinol encapsulated in the microspheres: 0.4%
b) Delmopinol in the mouthwash liquid phase: 0.2%

### EXAMPLE 3

### Preparation of a Mouthwash Containing S-Ibuprofen

### 3.1 Mouthwash Preparation

The composition of the polymer dispersion and of the cross-linking bath, prepared according to example 1, is reported in Table 6 below.

TABLE 6
Composition (grams) of the polymer dispersion and of the cross-linking bath

| | Composition of the dispersion | | | | Composition of the cross-linking | | | | |
|---|---|---|---|---|---|---|---|---|---|
| Mouthwash | Protanal | C | S- | | bath | | | | |
| n. | LF120M | 934P** | IB* | Water | CaCl₂ | S-IB* | CP⁺ | EtOH** | Water |
| 24 | 1.5 | 0.5 | 0.2 | 97.8 | 18.0 | 1.8 | 4 | 70 | 806.2 |

Notes:
*sodium S-ibuprofen;
⁺Cremophor EL;
**Ethanol

### 3.2 Determination of Aggregates Formation

After one-week storage, the mouthwash was stirred in order to re-suspend the microspheres, then 50 mL mouthwash was taken and filtered through a sieve (sieve opening: 710 μm). The presence of aggregates was evaluated according to example 1.2 above. The assay requirements were complied with (i.e. no aggregates were deemed to be present) when sieve weight variation was lower than 8 mg.

### Results

The assay requirements were complied with.

### 3.3 Content Assay

### Sample Preparation

### a) S-Ibuprofen Encapsulated in the Microspheres

20 ml mouthwash were filtered through a HVLP 0.45 membrane filter, and the microspheres on the filter were washed with 2 mL MeOH. The microspheres (about 50 mg) were dispersed in 10 mL sodium chloride (5 w/v) and digested by sonication for 1 hour. The sample was filtered through a 0.45 μm polypropylene filter and analyzed by HPLC with the method described below.

### b) S-Ibuprofen in the Mouthwash Liquid Phase

2 mL aliquot of the mouthwash was filtered through a polypropylene 0.45 μm filter, diluted 1:10 with mobile phase [acetonitrile/water/acetic acid (50/46/4 v/v/v)] and analysed by HPLC with the method described below.

### HPLC Method

HPLC-UV (HP1100 Chemstations, Hewlett Packard, USA); Column: C18 reverse-phase (Novapack, Waters, Ireland) 4.6 mm×150 mm; Flux: 1.5 mL/min; mobile phase: Acetonitrile/water/acetic acid (50/46/4 v/v/v); injection volume: 20 μl; wavelength: 264 nm

### Results

a) S-ibuprofen encapsulated in the microspheres: 0.2%
b) S-ibuprofen in the mouthwash liquid phase: 0.2%

### EXAMPLE 4

### Preparation of Mouthwashes Containing Chlorhexidine Gluconate

### 4.1 Mouthwash Preparation

The compositions of the polymer dispersions and of the cross-linking bath, prepared according to example 1, is reported in Table 7 below.

TABLE 7
Composition (grams) of the polymer compositions and of the cross-linking baths

| | Composition of the polymer dispersion | | | | | | | | |
|---|---|---|---|---|---|---|---|---|---|
| Mouthwash | Protanal | C | | Composition of the cross-linking bath | | | | | |
| n. | LF120M | 934P*** | Water | CaCl₂ | CG* | EtOH** | flavor | Ssc | water |
| 25(ex 21) | 1.5 | 0.5 | 98.0 | 18.0 | 5.4 | 1 | 1 | 0.1 | 875.5 |
| 26 | 1.5 | 0.5 | 98.0 | 18.0 | 10 | 10 | 1 | 0.1 | 860.9 |

Notes:
*Chlorhexidine gluconate 20%; flavor: mint; Ssc: sodium saccharine

### 4.2 Determination of Aggregates Formation

After one-week storage, the mouthwashes were stirred in order to suspend the microspheres, then 50 mL aliquots were taken and filtered through a sieve (sieve opening: 710 μm). The presence of aggregates was evaluated according to example 1.2 above. The assay requirements were complied with (i.e. no aggregates were deemed to be present) when sieve weight variation was lower than 8 mg.

### Result

The assay requirements were complied with.

### 4.3 Content Assay

The assay was performed on mouthwash no. 26.

### Sample Preparation

### a) Chlorhexidine Gluconate Encapsulated in the Microspheres

20 ml each mouthwash was filtered through a HVLP 0.45 μm membrane filter, and the microspheres on the filter were washed with 2 mL MeOH. Aliquots of about 50 mg were taken and digested by sonication for 1 hour in 10 mL sodium chloride (5% w/v). The resulting samples, filtered through a polypropylene 0.45 μm filter, were analyzed by using a Beckman DU-65 spectrophotometer at 252 nm.

### b) Chlorhexidine Gluconate in the Mouthwash Liquid Phase

2 mL aliquots of each mouthwash were filtered through a polypropylene 0.45 μm filter and analyzed using a Beckman DU-65 spectrophotometer at 252 nm.

### Results

a) Chlorhexidine gluconate encapsulated in the microspheres: 0.06%
b) Chlorhexidine gluconate in the mouthwash liquid phase: 0.04%

### EXAMPLE 5

### Preparation of Mouthwashes Containing Flurbiprofen

### 5.1 Mouthwashes Preparation

The compositions of the polymer dispersions and of the cross-linking baths, prepared according to example 1, are reported in Table 8 below.

TABLE 8
Compositions (grams) of the polymer dispersions and of the cross-linking baths

| | Composition of the polymer dispersion | | | | | | | | | | | | | |
|---|---|---|---|---|---|---|---|---|---|---|---|---|---|---|
| Mouthwash | Protanal | C | | | Composition of the cross-linking bath | | | | | | | | | |
| n. | LF120M | 934P | FB* | Water | CaCl₂ | FB* | CP⁺ | EtOH** | Gly | Sorb | Ssc | flavor | Pres*** | Water |
| 27 | 1.5 | 1.5 | 0.1 | 96.9 | 9.0 | 0.9 | 21.6 | 90 | 90 | 63 | 1.35 | 1.8 | 1.08 | 621.27 |
| 28 | 1.5 | 1.5 | 0.2 | 196.8 | 8.0 | 0.8 | 19.2 | 80 | 80 | 56 | 1.2 | 1.6 | 0.96 | 552.24 |

Notes:
*Flurbiprofen;
⁺Cremophor RH;
**Ethanol; gly: glycerol; sorb: sorbitol; Ssc: sodium saccharine; flavor: mint;
***preservative: Methyl-paraben.

### 5.2 Determination of Aggregates Formation

After one week-storage the mouthwashes were stirred in order to re-suspend the microparticles, then 50 mL aliquots were taken and filtered through a sieve (sieve opening: 710 μm). The presence of aggregates was evaluated according to example 1.2 above. The assay requirements were complied with (i.e. no aggregates were deemed to be present) when sieve weight variation was lower than 8 mg.

### Result

The assay requirements were complied with.

### 5.3 Content Assay

### Sample Preparation

### a) Flurbiprofen Encapsulated in the Microspheres

20 mL each mouthwash was filtered through a HVLP 0.45 μm membrane filter and about 50 mg of the microspheres remaining on the filter was digested by sonication for 1 hour in 10 mL of a NaCl (5% w/v)/MeOH (50/50 v/v) solution. The resulting samples were filtered through a polypropylene 0.45 μm filter and analyzed by HPLC, according to the method reported below.

### b) Flurbiprofen in the Mouthwash Liquid Phase

2 mL aliquots of each mouthwash were filtered through a polypropylene 0.45 μm filter, diluted 1:10 with a mobile phase made of MeOH/Phosphate buffer 0.01 M pH 2.5 (66/34 v/v), then analysed by HPLC, according to the method reported below.

### HPLC Method

HPLC-UV (HP1100 Chemstations, Hewlett Packard, USA); Column: ODS Hypersil 4.6 mm×100 mm, 3 μm (Thermo Scientific); flux: 1.2 mL/min; mobile phase: MeOH/Phosphate buffer 0.01 M pH 2.5 (66/34 v/v); injection volume: 10 μl; wavelength: 254 nm

### Results

### Mouthwash n. 27

a) Flurbiprofen encapsulated in the microspheres: 0.1%
b) Flurbiprofen in the mouthwash liquid phase: 0.1%

### Mouthwash n. 28

a) Flurbiprofen encapsulated in the microspheres: 0.3%
b) Flurbiprofen in mouthwash liquid phase: 0.06%

### EXAMPLE 6

### Preparation of Mouthwashes Containing Clobetasol Propionate

### 6.1 Mouthwash Preparation

The composition of the polymer dispersion and of the cross-linking bath, prepared according to example 1, is reported in Table 9.

TABLE 9
Composition (grams) of the polymer dispersion and of the cross-linking bath

| | Composition of the polymer dispersion | | | | | | | | | | | | | |
|---|---|---|---|---|---|---|---|---|---|---|---|---|---|---|
| Mouthwash | Protanal | C | | | Composition of the cross-linking bath | | | | | | | | | |
| n. | LF120M | 934P | ClP* | Water | CaCl₂ | ClP* | CP⁺ | EtOH** | Gly | Sorb | Ssc | Flavor | Pres*** | Water |
| 29 | 1.5 | 1.5 | 0.025 | 96.9 | 9.0 | 0.23 | 21.6 | 90 | 90 | 63 | 1.35 | 1.8 | 1.08 | 622 |

*Clobetasol propionate;
⁺Cremophor RH;
**Ethanol; gly: glycerol; sorb: sorbitol; Ssc: sodium saccharine; flavor: mint;
***preservative: Methyl-paraben.

### 6.2 Determination of Aggregates Formation

After one-week storage, the mouthwash was stirred to re-suspend the microparticles, then 50 mL aliquots were taken and filtered through a sieve (sieve opening: 710 μm). The presence of aggregates was evaluated according to example 1.2 above. The assay requirements were complied with (i.e. no aggregates were deemed to be present) when sieve weight variation was lower than 8 mg.

### Result

The assay requirements were complied with.

### EXAMPLE 7

### In Vitro Penetration Study

Mouthwash no. 27 was used to evaluate flurbiprofen penetration into buccal porcine mucosa. The performance of this mouthwash was compared to that of a commercially available mouthwash (Italian trade name Brufen® spray) containing 0.25 w/w active ingredient, i.e. 2.5-fold higher than that of formulation n. 27.

### Method

Apparatus—A three component in-house equipment consisting of: (a) six in-series mucosa supports; (b) a peristaltic pump and (c) a collector of fractions was used. The mucosa supports consisted of self-made disposable chamber sets at an acute angle of 30° to guarantee the efflux of pH 6.4 phosphate buffer (Na₂HPO₄ 1.79 g/l; KH₂PO₄ 1.36 g/l; NaCl 7.02 g/l), which was added dropwise at 1 ml/min to simulate the buccal environment and the flushing action by saliva. The buffer effused from the sample supports was collected in glass bottles.

Mucosa preparation—Fresh porcine cheek mucosa was dipped for min in pH 7.4 saline isotonic solution (KH₂PO₄ 1.90 g/l; Na₂HPO₄ 8.10 g/l; NaCl 4.11 g/l) at 70° C., and then the epithelium was peeled off the mucosa edges.

Penetration test—Mouthwash no. 27 and Brufen® spray were sprayed once by means of a buccal spray device onto 2.5 cm×1 cm mucosal epithelia, prepared as described above. The amount of formulation delivered onto the epithelia was about 80 mg. The epithelia were then placed on suitable supports, sacrificed at predefined times (1, 3 and 6 hours) and the tested mouthwashes present on their surface were peeled off by means of an adhesive tape strip. The epithelia were then stored at −40° C. for 24 hours, homogenized and the amount of penetrated ibuprofen was extracted with 5 ml methanol and measured.

### Result

The amount of flurbiprofen penetrated into the porcine mucosa as a function of time is reported in table 10 below

TABLE 10

| | | Formulation |
|---|---|---|
| Time | Froben | no 27 |
| (h) | (ng/mg) | (ng/mg) |
| 1 | 84 ± 17 | 48 ± 9 |
| 3 | 24 ± 9 | 56 ± 26 |
| 6 | 5 ± 2 | 9 ± 4 |

The results show that the mouthwash of the invention ensured a penetration flurbiprofen comparable to that of a commercially available mouthwash having a flurbiprofen content 2.5-fold higher.

1. A mouthwash comprising microspheres incorporating an active ingredient wherein the microspheres are obtained in situ by adding drops of size smaller than 700 μm of an aqueous polymer dispersion (A) containing:
a) polyacrylic acid, optionally cross-linked and optionally in the form of a salt with an alkali metal; and
b) an alkali metal alginate
to an aqueous solution (B) containing a pharmaceutically acceptable organic or inorganic salt of an alkaline earth metal able to cross-link the alginate, wherein the active ingredient (c) is contained either in polymer dispersion (A) or in aqueous solution (B) or in both dispersion (A) and solution (B).

2. The mouthwash according to claim 1, wherein polymer total content in polymers (a)+(b) ranges from 0.5 to 5% by weight with respect to the mouthwash total weight.

3. The mouthwash according to claim 1, with an alkali metal alginate/optionally cross-linked polyacrylate weight to weight ratio ranging from 1:1 to 14:1.

4. The mouthwash according to claim 1, in which the alkali metal alginate is sodium alginate with a viscosity ranging from 10 to 400 mPa·s, as measured in a solution containing said alginate in 1% concentration at 20° C.

5. The mouthwash according to claim 1, in which the polymer dispersion (A) is added to the aqueous solution (B) in amounts ranging from 5 to 25% by weight with respect to the mouthwash total weight.

6. The mouthwash according to claim 1, in which the microsphere size is smaller than 700 μm.

7. The mouthwash according to claim 6, wherein the particle size distribution of the microspheres present in the mouthwash, measured as volume d₉₀, ranges from 50 to 500 μm.

8. The mouthwash according to claim 1, wherein the active ingredient is selected from the group consisting of local anaesthetics, nonsteroidal antiinflammatory drugs, corticosteroids, antiseptics, anticaries agents, and cicatrizants.

9. The mouthwash according to claim 8, in which the microspheres contain the active ingredient in amounts ranging from 0.01 to 5% by weight with respect to the microspheres total weight.

10. A process for preparing the mouthwash according to claim 1, comprising the following steps:
i) dispersing in water an optionally cross-linked polyacrylic acid (a) or an alkaline salt thereof and an alkali metal alginate (b), in order to obtain an aqueous polymer dispersion (A)
ii) spraying drops of size smaller than 700 μm of the aqueous polymer dispersion (A), obtained in the previous step, into an aqueous solution (B) including a pharmaceutically acceptable organic or inorganic salt of an alkali metal able to cross-link the alginate
wherein the active ingredient (c) is added either to polymer dispersion (A) or to aqueous solution (B) or to both dispersion (A) and solution (B).
